(19) United States
(12) Plant Patent Application Publication
(10) Pub. No.: US 2006/0107429 P1
(43) Pub. Date: May 18, 2006

(54) VIBURNUM PLANT NAMED 'C.A. HILDEBRANT'S'

(50) VIBURNUM WRIGHTII; VIBURNUM

(76) Inventor: Charles A. Hildebrant JR., Oldwick, NJ (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/989,322

(22) Filed: Nov. 17, 2004

(51) Int. Cl. A01H 5/00

(52) U.S. Cl. PLT/226

(57) ABSTRACT

The new Viburnum wrightii cultivar is provided that was created through an extensive controlled crossing and selection program using unreleased and unnamed parent plants. The growth habit is very dense and compact. Attractive dense relatively flat clusters of white flowers commonly are produced during mid-May to early June. Also, attractive red berries of unusually long persistence are formed. The cold hardiness is good. The new cultivar is well suited to provide attractive colorful ornamentation in gardens and in the landscape over an extended period of time.

### BOTANICAL/COMMERCIAL CLASSIFICATION

Viburnum wrightii/Viburnum Plant

### VARIETAL DENOMINATION

cv. ‘C. A. Hildebrant's’

### SUMMARY OF THE INVENTION

Viburnum plants are recognized to be a varied and diversified group of shrubs that are widely grown to provide ornamentation. They are appreciated for their white, cream and sometimes pink spring flowers, good foliage, interesting shape, colored fruit that attracts birds, and their showy autumn color.

A new and distinct Wright Viburnum (i.e., Viburnum wrightii) plant is provided that is the product of the controlled crossing and selection program of Viburnum wrightii plants (unreleased, unnamed, and non-patented in the United States) at Oldwick, N.J., U.S.A., that was begun in the mid-1940's. Throughout the program the progeny were studied and were evaluated primarily for consistent berry set, growth habit, and flower production. This program resulted in the production of fifteen field grown plants from which a single plant as described herein was ultimately selected.

It was found that the new Viburnum wrightii plant of the present invention possesses the following combination of characteristics:
(a) displays a very dense and compact growth habit,
(b) produces in abundance during mid-May to early-June attractive dense relatively flat clusters of white flowers,
(c) forms attractive red berries of long persistence, and
(d) displays good cold hardiness.

The new cultivar of the present invention can be readily distinguished from other Viburnum wrightii plants in view of the recited combination of characteristics. The brilliant fall foliage coloration and the long lasting berry set are particularly noteworthy and provide additional seasonal interest. In the fall the foliage commonly turns red, amber and maroon. The plant has well withstood temperatures as low as −10° F. without harm.

The new cultivar is well suited to provide attractive colorful ornamentation in gardens and in the landscape over an extended period of time.

Asexual reproduction of the new cultivar at Oldwick, N.J., U.S.A., by the rooting of cuttings has demonstrated that the distinctive characteristics of the new cultivar of the present invention are reliably transmitted from one generation to another. Accordingly, the new cultivar reproduces in a true-to-type manner by such technique.

The new cultivar has been named ‘C. A. Hildebrant's’.

### BRIEF DESCRIPTION OF THE PHOTOGRAPHS

The accompanying photographs show as true as it is reasonably possible to make the same in color illustrations of this character typical specimens of the plant and plant parts of the new cultivar of the present invention. The depicted plants were approximately four years of age and were growing outdoors under full sun at West Grove, Pa., U.S.A.

FIG. 1 shows an overall flowering plant of the new cultivar wherein the very dense and compact growth habit and the abundant presence of attractive dense relatively flat clusters of white flowers are shown.

FIG. 2 shows a close view of the abundant presence of bright red berries and the foliage during early fall.

FIG. 3 shows a close enlarged view of a typical leaf during early fall. Some bright fall coloration has begun to appear primarily on a leaf in the background at the upper left portion of the photograph.

### DETAILED DESCRIPTION

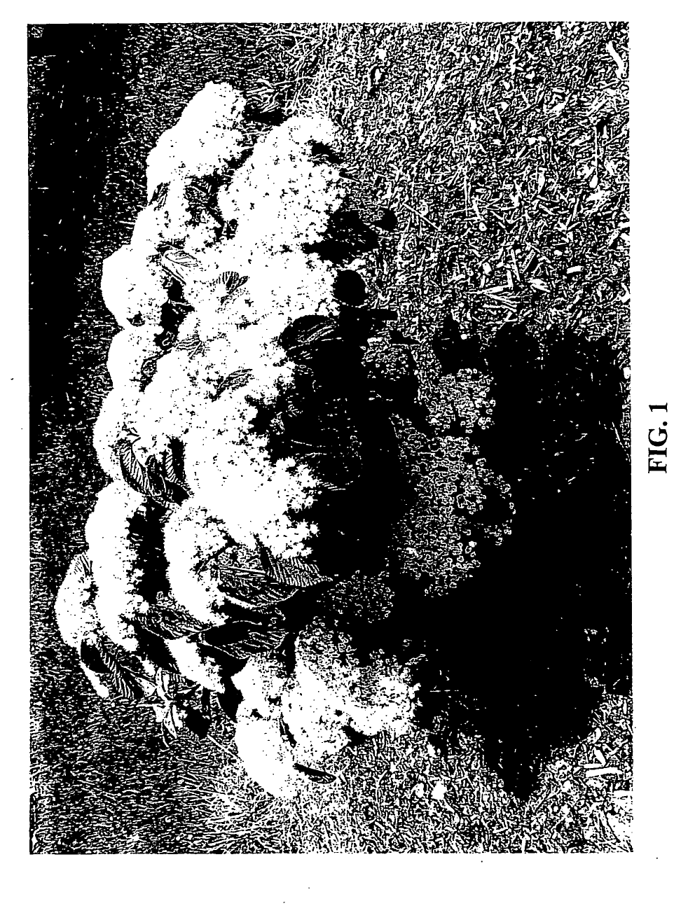

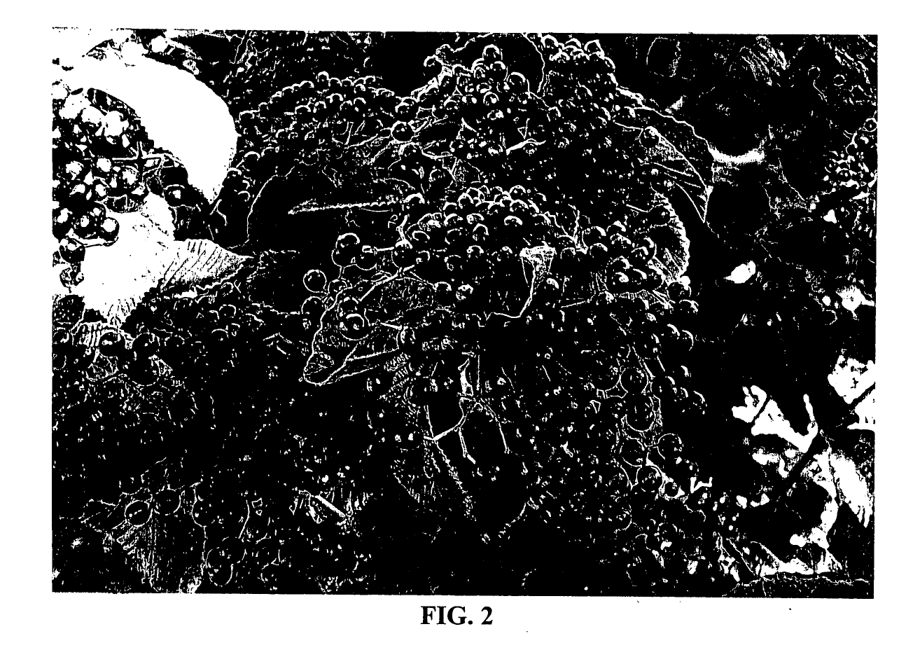

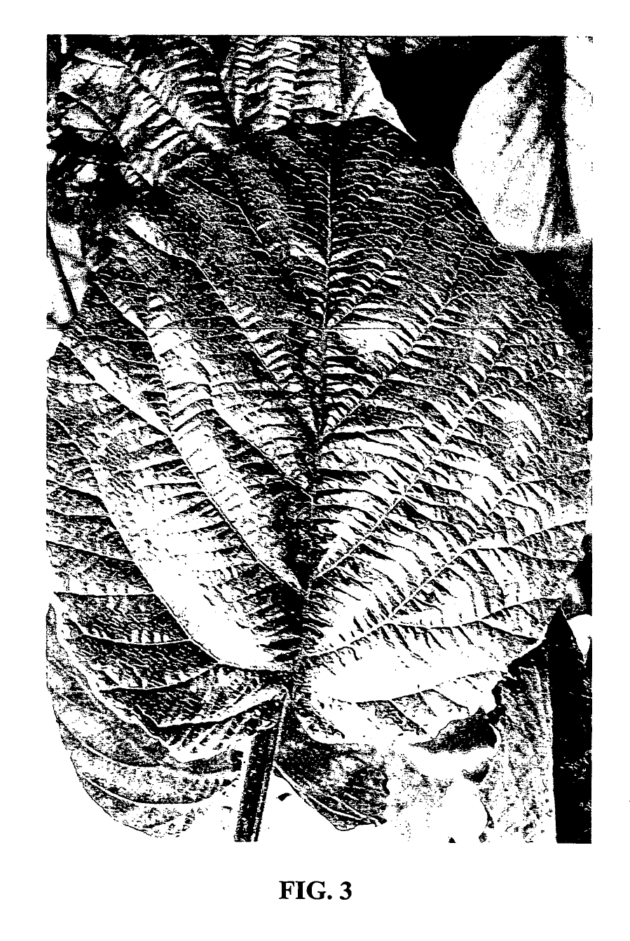

The following description is based upon the observation of mature plants of the new cultivar while growing in an outdoor garden in full sun at West Grove, Pa., U.S.A. The chart used in the identification of the colors is the R.H.S. Colour Chart of The Royal Horticultural Society, London, England.
Plant:
   Height.—Approximately 4 to 5 feet.
   Width.—Approximately 3 feet.
   Growth habit.—Very dense and compact.
   Branches.—Slender, smooth, Grey-Brown Group 199C in coloration, and commonly with approximately 4 or 5 lenticels per cm.
Foliage:
   Arrangement.—Opposite, single.
   Quantity.—Abundant.
   Mature length.—Approximately 13 cm on average.
   Mature width.—Approximately 8 cm at the widest point on average.
   Shape.—Broad-elliptic as illustrated in FIG. 3.
   Apex.—Generally obtuse.
   Base.—Cordate.
   Margin.—With slight crenation as illustrated in FIG. 3.
   Venation Pattern.—Commonly with 6 to 7 vein pairs as illustrated in FIG. 3.
   Texture.—Glabrous and glossy on the upper surface.
   Thickness.—Medium.
   Summer color.—Mature leaf: Upper surface: Green Group 131A. Under surface: Green Group 138A.
   Fall Color.—Commonly a brilliant blend of red, amber, and maroon in October that is maintained well into November.
   Petiole.—Approximately 2 cm in length.
   Glands.—None observed.
   Stipules.—None observed.
Inflorescence:
   Description.—In abundance in relatively flat clusters of compound cymes with approximately 20 to 25 flowers per cyme that are reminiscent of Queen Ann's Lace as illustrated in FIG. 1.
   Flowering time.—Commonly mid-May to early June. A first bloom has been observed on May 15^{th} with full bloom on June 5^{th}.
   Longevity.—Approximately 3 weeks.
   Quantity.—The number varies with the size and age of the plant. The flowers per inflorescence commonly number approximately 240 on young plants and approximately 550 on mature plants.
   Fragrance.—Sweet and musty.
   Sepals.—None observed.
   Buds.—Shape: narrow-pointed. — Size: approximately 0.5 cm in length and approximately 0.2 cm in width. — Color: commonly between White Group 155A and 155B.
   Flower diameter.—Relatively flat compound cymes in a cluster commonly measuring approximately 9.5 cm in diameter.
   Petal number.—Five fused petals.
   Petal shape.—Ovate.
   Petal length.—Approximately 3 mm.
   Petal width.—Approximately 2 mm.
   Petal color.—Near White Group 155D on both surfaces.
   Stamen.—Five in number, and approximately 5 mm in length.
   Filaments.—Approximately 4.2 mm in length and White Group 155D in coloration.
   Anthers.—Approximately 0.8 mm in size, and regularly arranged around the style.
   Pollen.—Commonly formed in each flower in a minute quantity and Yellow-Orange Group 22A in coloration.
   Pistil.—One per flower.
   Style.—Columnar, short and approximately 3.2 mm in length, and White Group 155D in coloration.
Fruit:
   Bearing onset.—Commonly September to late October.
   Quantity.—Moderately abundant as illustrated in FIG. 2.
   Size.—Generally round and approximately 7 mm in diameter.
   Color.—When ripe bright red, Red Group 46B, and when fully mature darkening to near Red Group 53A.
   Persistence.—The berries commonly are retained on the plant for six months or more and generally longer than previously known plants of the species.

The new cultivar has been found to grow well in U.S.D.A. Hardiness Zone No. 6.

No particular disease or pest problem has been encountered during observations to date.

The new ‘C. A. Hildebrant's’ cultivar has not been observed to date under all possible environmental conditions. Accordingly, it is possible that the phenotype may vary somewhat with variations in the environment, such as temperature, light intensity, day length, and other cultural conditions without variance of the genotype. For instance, leaf coloration may vary with the composition and concentration of the fertilizer that is utilized.

I claim:

1. A new and distinct Viburnum wrightii plant having the following combination of characteristics:
(a) displays a very dense and compact growth habit,
(b) produces in abundance during mid-May to early-June attractive dense relatively flat clusters of white flowers,
(c) forms attractive bright red berries of long persistence, and
(d) displays good cold hardiness; substantially as illustrated and described.
